(19) United States
(12) Patent Application Publication
(10) Pub. No.: US 2025/0064509 A1
(43) Pub. Date: Feb. 27, 2025

(54) WIDE-AREA FOCAL ABLATION CATHETER HAVING INSULATED PORTIONS

(71) Applicant: Boston Scientific Scimed, Inc., Maple Grove, MN (US)

(72) Inventors: Raju Viswanathan, Palo Alto, CA (US); Gary Long, Cincinnati, OH (US); Brendan Early Koop, Ham Lake, MN (US)

(21) Appl. No.: 18/810,160

(22) Filed: Aug. 20, 2024

Related U.S. Application Data
(60) Provisional application No. 63/578,585, filed on Aug. 24, 2023.

(51) Int. Cl. A61B 18/14; A61B 18/00

(52) U.S. Cl. CPC A61B 18/1492; A61B 2018/00077; A61B 2018/00083; A61B 2018/00267; A61B 2018/00351; A61B 2018/00577; A61B 2018/00613; A61B 2018/1467

(57) ABSTRACT

An electroporation catheter for ablation of cardiac tissue is disclosed. The electroporation catheter includes an elongated shaft having a proximal end and an opposite distal end. The elongated shaft defines an axis. An electrode assembly is coupled to and extends distally from the distal end of the elongated shaft. The electrode assembly is transitionable between a collapsed state and an expanded state. The electrode assembly includes conductive struts that define a spherical shape in the expanded state having a proximal portion coupled to the elongated shaft, a central portion having a maximum radial dimension and a distal portion opposite the elongated shaft. A proximal insulation portion is disposed on the proximal portion of the electrode assembly surrounding each of the plurality of conductive struts. The proximal insulator portion extends from the proximal end to at least the central portion.

### CROSS REFERENCE TO RELATED APPLICATIONS

This application claims priority to U.S. Provisional Application No. 63/578,585 entitled “WIDE-AREA FOCAL ABLATION CATHETER HAVING INSULATED PORTIONS,” filed Aug. 24, 2023, which is incorporated herein by reference in its entirety.

### TECHNICAL FIELD

The present disclosure relates to medical systems and methods for facilitating ablation of tissue in a patient. More specifically, the present disclosure relates to medical systems and methods for facilitating ablation of tissue by electroporation.

### BACKGROUND

Ablation procedures are used to treat many different conditions in patients. Ablation can be used to treat cardiac arrhythmias, benign tumors, cancerous tumors, and to control bleeding during surgery. Usually, ablation is accomplished through thermal ablation techniques including radio-frequency (RF) ablation and cryoablation. In RF ablation, a probe is inserted into the patient and radio frequency waves are transmitted through the probe to the surrounding tissue. The radio frequency waves generate heat, which destroys surrounding tissue and cauterizes blood vessels. In cryoablation, a hollow needle or cryoprobe is inserted into the patient and cold, thermally conductive fluid is circulated through the probe to freeze and kill the surrounding tissue. RF ablation and cryoablation techniques can indiscriminately kill tissue through cell necrosis, which may damage or kill otherwise healthy tissue, such as tissue in the esophagus, phrenic nerve cells, and tissue in the coronary arteries.

Another ablation technique uses electroporation. In electroporation, or electro-permeabilization, an electrical field is applied to cells in order to increase the permeability of the cell membrane. The electroporation can be reversible or irreversible, depending on the strength and duration of the electric field. If the electroporation is reversible, the temporarily increased permeability of the cell membrane can be used to introduce chemicals, drugs, or deoxyribonucleic acid (DNA) into the cell, prior to the cell healing and recovering. Tissue recovery can occur over minutes, hours, or days after the ablation is completed. If the electroporation is irreversible, the affected cells are killed, such as via form of cell death, such as perhaps programmed cell death through apoptosis for example, or such as traumatic cell death through necrosis for example.

Irreversible electroporation can be used as a nonthermal ablation technique. In irreversible electroporation, trains of short, high voltage pulses are used to generate electric fields that are strong enough to kill cells. In ablation of cardiac tissue, irreversible electroporation can be a relatively safe and effective alternative to the indiscriminate killing of thermal ablation techniques, such as RF ablation and cryoablation. Irreversible electroporation can be used to kill targeted tissue, such as myocardium tissue, by using a selected electric field strength and duration that is effective to kill the targeted tissue but is not effective to permanently damage other cells or tissue, such as non-targeted myocardium tissue, red blood cells, vascular smooth muscle tissue, endothelium tissue, and nerve cells.

### SUMMARY

In Example 1, an electroporation catheter for ablation of cardiac tissue. The electroporation catheter comprising: an elongated shaft having a proximal end and an opposite distal end, the elongated shaft defining an axis; an electrode assembly coupled to and extending distally from the distal end of the elongated shaft, the electrode assembly transitionable between a collapsed state and an expanded state wherein the electrode assembly includes a plurality of conductive struts defining a spherical shape in the expanded state having a proximal portion coupled to the elongated shaft, a central portion having a maximum radial dimension and a distal portion opposite the elongated shaft; and a proximal insulation portion disposed on the proximal portion of the electrode assembly surrounding each of the plurality of conductive struts, the proximal insulator portion extending from the proximal end to at least the central portion.

In Example 2, the electroporation catheter of Example 1, wherein the ablation electrode in the expanded state includes maximum radial dimension greater than a maximum radial dimension of the distal region of the elongated shaft.

In Example 3, the electroporation catheter of Example 2, wherein the maximum radial dimension of the ablation electrode defines an equator on the electrode assembly.

In Example 4, the electroporation catheter of Example 3, wherein the proximal insulation is disposed on the conductive struts from the proximal end to the equator.

In Example 5, the electroporation catheter of Example 3, wherein the proximal insulator is disposed on the conductive struts from the proximal end to a chord on the electrode assembly distal to the equator.

In Example 6, the electroporation catheter of any of Examples 1-5, wherein the conductive struts form a lattice hull.

In Example 7, the electroporation catheter of Example 6, wherein the conductive struts are arranged in a plurality of cells on the lattice hull.

In Example 8, the electroporation catheter of any of Examples 1-5, wherein the conductive struts form a plurality of longitudinally extending splines to form a basket hull.

In Example 9, the electroporation catheter of any of Examples 1-8, further comprising a shaft electrode disposed on the distal region of the shaft.

In Example 10, the electroporation catheter of Example 9, wherein the electrode assembly and shaft electrode are configured as one of a cathode and an anode to generate an electric field in a monopolar mode.

In Example 11, the electroporation catheter of Example 9, wherein the electrode assembly is configured as one of a cathode and an anode and the shaft electrode is configured as the other of the anode and the cathode to generate an electric field in a bipolar mode.

In Example 12, the electroporation catheter of any of Examples 1-11, further comprising a plurality of sense electrodes disposed on the electrode assembly.

In Example 13, the electroporation catheter of Example 12, wherein the electrode assembly includes a plurality of electrodes configured as one of a cathode and an anode to generate an electric field in a monopolar mode.

In Example 14, the electroporation catheter of any of Examples 1-13, further comprising a second insulator selectively disposed on the conductive struts distal to the proximal insulation portion.

In Example 15, the electroporation catheter of Example 14, wherein the second insulator is disposed on an inner surface of a strut of the plurality of conductive struts facing an internal cavity of the electrode assembly, and an outer surface of the strut of the plurality of conductive struts is exposed.

In Example 16, an electroporation catheter for ablation of cardiac tissue. The electroporation catheter comprising: an elongated shaft having a proximal end and an opposite distal end, the elongated shaft defining an axis; an electrode assembly coupled to and extending distally from the distal end of the elongated shaft, the electrode assembly transitionable between a collapsed state and an expanded state wherein the electrode assembly includes a plurality of conductive struts defining a spherical shape in the expanded state having a proximal portion coupled to the elongated shaft, a central portion having a maximum radial dimension and a distal portion opposite the elongated shaft; and a proximal insulation portion disposed on the proximal portion of the electrode assembly surrounding each of the plurality of conductive struts, the proximal insulator portion extending from the proximal end to at least the central portion.

In Example 17, the electroporation catheter of Example 16, wherein the ablation electrode in the expanded state includes maximum radial dimension greater than a maximum radial dimension of the distal region of the elongated shaft.

In Example 18, the electroporation catheter of Example 17, wherein the maximum radial dimension of the ablation electrode defines an equator on the electrode assembly.

In Example 19, the electroporation catheter of Example 18, wherein the proximal insulation is disposed on the conductive struts from the proximal end to the equator.

In Example 20, the electroporation catheter of Example 18, wherein the proximal insulator is disposed on the conductive struts from the proximal end to a chord on the electrode assembly distal to the equator.

In Example 21, the electroporation catheter of Example 16, wherein the conductive struts form a lattice hull.

In Example 22, the electroporation catheter of Example 21, wherein the conductive struts are arranged in a plurality of cells on the lattice hull.

In Example 23, the electroporation catheter of Example 16, wherein the conductive struts include a plurality of longitudinally extending splines to form a basket hull.

In Example 24, the electroporation catheter of Example 16, further comprising a shaft electrode disposed on the distal region of the shaft.

In Example 25, the electroporation catheter of Example 24, wherein the electrode assembly and shaft electrode are configured as one of a cathode and an anode to generate an electric field in a monopolar mode.

In Example 26, the electroporation catheter of Example 24, wherein the electrode assembly is configured as one of a cathode and an anode and the shaft electrode is configured as the other of the anode and the cathode to generate an electric field in a bipolar mode.

In Example 27, the electroporation catheter of Example 16, further comprising a plurality of sense electrodes disposed on the electrode assembly.

In Example 28, the electroporation catheter of Example 27, wherein the electrode assembly includes a plurality of electrodes configured as one of a cathode and an anode to generate an electric field in a monopolar mode.

In Example 29, the electroporation catheter of Example 16, further comprising a second insulator selectively disposed on the conductive struts distal to the proximal insulation portion.

In Example 30, the electroporation catheter of Example 29, wherein the second insulator is disposed on an inner surface of a strut of the plurality of conductive struts facing an internal cavity of the electrode assembly, and an outer surface of the strut of the plurality of conductive struts is exposed.

In Example 31, an electrophysiology system comprising an electroporation console configured to generate pulsed electrical signals for electroporation ablation and an electroporation catheter coupled to the electroporation console. The electroporation catheter comprising: an elongated shaft having a proximal end and an opposite distal end, the elongated shaft defining an axis; an electrode assembly coupled to and extending distally from the distal end of the elongated shaft, the electrode assembly transitionable between a collapsed state and an expanded state wherein the electrode assembly includes a plurality of conductive struts defining a spherical shape in the expanded state having a proximal portion coupled to the elongated shaft, a central portion having a maximum radial dimension and a distal portion opposite the elongated shaft; and a proximal insulation portion disposed on the proximal portion of the electrode assembly surrounding each of the plurality of conductive struts, the proximal insulator portion extending from the proximal end to at least the central portion.

In Example 32, the electroporation catheter of Example 31, wherein the electroporation catheter is configured to be operated in a bipolar mode and a monopolar mode.

In Example 33, an electroporation catheter for ablation of cardiac tissue. The electroporation catheter comprising: an elongated shaft having a proximal end and an opposite distal end, the elongated shaft defining an axis and including a shaft electrode disposed on the distal end; an electrode assembly coupled to and extending distally from the distal end of the elongated shaft, the electrode assembly transitionable between a collapsed state and an expanded state wherein the electrode assembly includes a plurality of conductive struts defining a spherical shape in the expanded state having a proximal portion coupled to the elongated shaft, a central portion having a maximum radial dimension and a distal portion opposite the elongated shaft, the electrode assembly operable in a bipolar mode and a monopolar mode; and a proximal insulation portion disposed on the proximal portion of the electrode assembly surrounding each of the plurality of conductive struts, the proximal insulator portion extending from the proximal end to at least the central portion. The electrode assembly and shaft electrode are configured as one of a cathode and an anode to generate an electric field in the monopolar mode. The electrode assembly is configured as one of a cathode and an anode and the shaft electrode is configured as the other of the anode and the cathode to generate an electric field in the bipolar mode.

In Example 34, the electroporation catheter of Example 33, wherein the ablation electrode in the expanded state includes maximum radial dimension greater than a maximum radial dimension of the distal region of the elongated shaft, and wherein the proximal insulator is disposed on the conductive struts from the proximal end to a chord on the electrode assembly distal to the maximum radial dimension.

In Example 35, the electroporation catheter of Example 33, wherein the conductive struts form one of a lattice hull and a basket hull.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a patient, and for treating a heart of the patient, using an electrophysiology system, in accordance with embodiments of the subject matter of the disclosure.

FIG. 2 is a schematic diagram illustrating a distal portion of an example catheter that can be used with the example electrophysiology system of FIG. 1.

FIGS. 3A-3B illustrate example distal portions of catheters in accordance with the example catheter of FIG. 2.

FIGS. 4A-4B illustrate example distal portions of catheters in accordance with the example catheter of FIG. 2.

FIGS. 5A and 5B illustrate example electric fields generated via the example catheter of FIG. 2.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) of the features in an example used across all examples. Thus, no one figure should be interpreted as having any dependency or requirement related to any single component or combination of components illustrated therein. Additionally, various components depicted in a figure may be, in examples, integrated with various ones of the other components depicted therein (or components not illustrated), all of which are considered to be within the ambit of the present disclosure.

As applied to electrophysiology systems, irreversible electroporation uses high voltage, short pulses to kill cells such as myocardium through apoptosis while sparing other adjacent tissues including the esophageal vascular smooth muscle and endothelium. Irreversible electroporation treatment can be delivered in multiple therapy sections. A therapy section, which may have a duration on the order of milliseconds, may include a plurality of electrical pulses, such as a few dozen pulses, generated and delivered by an electroporation device, which is powered by an electroporation generator, to generate an electric field of sufficient strength to create transmural lesions. Such pulsed field ablation creates generally spherical electric fields around a generally point electrode, or electric fields having generally circular cross sections around a generally linear electrode, that is generally independent of tissue or blood in the heart. Such fields can include the potential to cause collateral damage to adjacent areas on the blood pools side of the electrode, or side opposite to the target tissue. In contrast, RF ablation can be focused to areas in direct contact with the electrode.

Electroporation devices that perform ablation and cardiac mapping, such as mapping and ablation catheters, generally are of a larger size than many RF ablation catheters. For example, a working end of a mapping and ablation catheter may be 7 mm to 15 mm or more in diameter. Such catheters often produce relatively large electric fields for ablation, which increase the potential for collateral damage to areas opposite the myocardial target tissue.

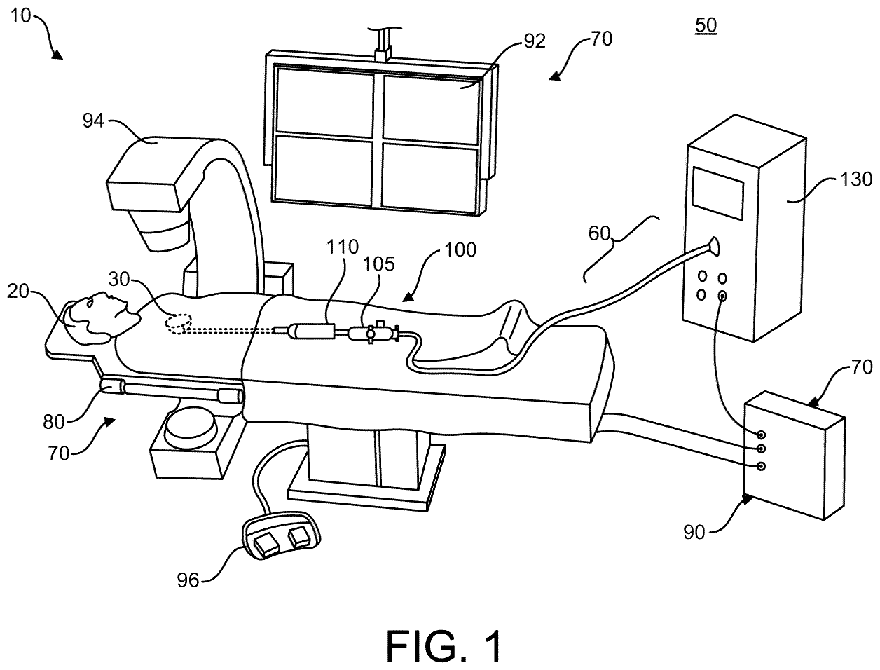

FIG. 1 illustrates an example clinical setting 10 for treating a patient 20, such as for treating a heart 30 of the patient 20, using an electrophysiology system 50, in accordance with the disclosure. The electrophysiology system 50 includes an electroporation catheter system 60 and an electro-anatomical mapping (EAM) system 70. The example electroporation catheter system 60 includes an electroporation catheter 105, an introducer sheath 110, and an electroporation console 130. Additionally, the electroporation catheter system 60 includes various connecting elements, such as cables, that operably connect the components of the electroporation catheter system 60 to one another and to the components of the EAM system 70. In general, the EAM mapping system 70 includes a localization field generator 80, a mapping and navigation controller 90, and a display 92. Also, the clinical setting 10 can include additional equipment such as imaging equipment 94 (represented by the C-arm) and various controller elements, such as a foot controller 96, configured to allow an operator to control various aspects of the electrophysiology system 50. The clinical setting 10 may have other components and arrangements of components that are not shown in FIG. 1.

The electroporation catheter system 60 is configured to deliver ablation electric field energy to targeted tissue in the patient's heart 30 to create cell death in tissue, for example, rendering the tissue incapable of conducting electrical signals. Also, the electroporation catheter system 60 is configured to generate electric fields using the electroporation catheter 105 to create and present, on the display 92, an electro-anatomical map of the patient's heart to aid a clinician in planning ablation by irreversible electroporation using the electroporation catheter 105 prior to delivering ablation electric field energy. In embodiments, the electroporation catheter system 60 is configured to generate the electric fields based on characteristics of the electroporation catheter 105 and the position of the electroporation catheter 105 in the patient 20, such as in the heart 30 of the patient 20. The electroporation catheter system 60 is configured to generate graphical representations of the electroporation catheter and the electro-anatomical map based on characteristics of the electroporation catheter 105 and the position of the electroporation catheter 105 in the patient 20, such as in the heart 30 of the patient 20, and the characteristics of the tissue surrounding the catheter 105, such as measured impedances of the tissue. In one example, the electroporation catheter 105 is a mapping and ablation catheter, which can be deployed in mapping procedures in cooperation with the EAM system 70 as well as to deliver ablation electric field energy and ablate tissue via irreversible electroporation.

The introducer sheath 110 is operable to provide a delivery conduit through which the electroporation catheter 105 can be deployed to the specific target sites within the patient's heart 30. Access to the patient's heart can be obtained through a vessel, such as a peripheral artery or vein. Once access to the vessel is obtained, the electroporation catheter 105 can be navigated to within the patient's heart, such as within a chamber of the heart.

The example electroporation catheter 105 includes an elongated catheter shaft and distal end region configured to be deployed proximate target tissue, such as within a chamber of the patient's heart. The shaft can extend from an access point in the patient to the target tissue and generally defines a longitudinal axis of the electroporation catheter 105. The distal end region may include a basket, balloon, spline, configured tip, or other electrode deployment mechanism coupled to the shaft. The electrode deployment mechanism includes an electrode assembly, or array, comprising an electrode. For example, the electrode assembly can include a plurality of spaced-apart electrodes or multiple spaced-apart sets or groups of spaced-apart electrodes. In some examples, an electrode, such as a plurality of spaced-apart electrodes, can be deployed on the catheter shaft in addition to or instead of an electrode on the electrode deployment mechanism. For instance, the electrode deployment mechanism includes a plurality of splines configured to form a basket, and at least a some of the electrodes are disposed on the splines.

The electroporation catheter 105 is configurable in a plurality of configurations. For example, when the distal end region of the catheter 105 is within a sheath as a catheter assembly, such as to travel to the patient to the chamber of the heart, the electrode deployment mechanism and electrode assembly are in a collapsed state to fit within the sheath. Once the catheter has reached the destination in the chamber of the heart, for example, the sheath is retracted from the distal region of the catheter 105 (or the shaft catheter is extended past the sheath) and the electrode deployment mechanism and electrode assembly can be arranged in an expanded state. The electrode assembly has a collapsed shape when the catheter 105 is in the collapsed state and an expanded shape when the catheter 105 is in the expanded state. In some examples, the electrode assembly has more than two states.

In one example, the plurality of electrodes can be formed of a conductive, solid-surface, biocompatible material and are spaced-apart across insulators. Each of the plurality of electrodes is electrically coupled to a corresponding elongated lead conductor that extend along the shaft to a catheter proximal end. In one example, each electrode of the spaced-apart electrodes corresponds with a separate, single lead conductor. In another example, a plurality of electrodes may be coupled to a single lead conductor. Other configurations are contemplated. The plurality of lead conductors can be insulated from one another within an insulating sheath along the catheter shaft, such as with an insulating polymer sheath. The lead conductors can be electrically coupled to plug in the proximal region of the electroporation catheter 105, such as a plug configured to be mechanically and electrically coupled to the electroporation console 130, for example, either directly or via intermediary electrical conductors such as cabling.

The electroporation console 130 includes a controller, such one or more controllers, processors, or computers, that executes instructions or code, such as processor-executable instructions, out of a non-transitory computer readable medium, such as a memory device, or memory, to cause, such as control or perform, the aspects of the electroporation catheter system 60. In one example, the electroporation console 130 is configured to provide an electrical signal, such as a plurality of concurrent or space-apart-time electrical signals, to the electrically connected electroporation catheter 105 along lead conductors to the spaced-apart electrodes. The spaced-apart electrodes are configured to generate a selected electrical field proximate the target tissue, based on the electrical signals from the electroporation console 130, such as to effect ablation.

The electroporation console 130 can generate electrical signals and select which electrodes in the electrode array will receive the electrical signals. A first electrode, or first group of electrodes, can be selected to be an anode and a different, second electrode, or second group of electrodes, can be selected to be a cathode, such that electrical fields can be generated between the anode and cathode based on signals, such as pulses, provided to the electrodes from the electroporation console 130. The console 130 provides electric pulses of different lengths and magnitudes to the electrodes on the catheter 105. The electric pulses can be provided in a continuous stream of pulses or in multiple, separate trains of pulses. Pulse parameters of interest include the number of pulses, the duty cycle of the pulses, the spacing of pulse trains, the voltage or magnitude of the pulses including the peak voltages, and the duration of the voltages. For example, the console 130 can select two or more electrodes of the electrode assembly and provides pulses to the selected electrodes to generate electric fields between the selected electrodes.

In an ablation mode, the console can select electrodes to provide pulsed field ablation (PFA). For example, PFA can be performed with monophasic waveforms and biphasic waveforms. Without being bound to a particular theory, electric field strengths in the range of generally 200-250 volts per centimeter (V/cm) with microsecond-scale pulse duration have been demonstrated to provide reversible electroporation in cardiac tissue. Electric field strengths at approximately 400 V/cm have been demonstrated to provide irreversible electroporation in cardiac tissue of interest, such as targeted myocardium tissue and endocardium tissue, with demonstrable sparing of red blood cells, vascular smooth muscle tissue, endothelium tissue, nerves and other non-targeted proximate tissue.

Additionally, the electrode assembly on catheter 105 can be operated in a selected mode such as monopolar mode or bipolar mode. During monopolar operation of the catheter 105, an electrode, a group of electrodes, or the entire electrode assembly are configured as one of an anode or a cathode. None of the electrodes in the electrode assembly are configured as a the other of the cathode or the anode. Instead, the other of the cathode or the anode is provided in the form of a pad dispersive electrode located on the patient, typically on the back, buttocks, or other suitable anatomical location during electroporation. An electrical field is formed between an activated electrode of the electrode assembly and the pad dispersive electrode. In an alternative configuration, a return electrode, such as a plurality of return electrodes, can be disposed on the shaft of the catheter. An electrical field is formed between an activated electrode of the electrode assembly and the return electrode on the shaft of the catheter 105. During bipolar operation of the catheter 105, a first set of one or more electrodes of the electrode assembly, is configured as the anode and a second set of one or more electrodes of the electrode assembly, is configured as the cathode, to generate the electric field. In this example, a pad dispersive electrode is not used, and the electrical field is not extended in the patient's body, but rather through a localized portion of tissue proximate the electrode assembly.

In some embodiments, the catheter can include sensing electrodes to receive electrical signals from the target tissue rather than to deliver electrical signals. For example, the catheter 105 can be a mapping and ablation catheter, and the electrodes can include ablation electrodes that are configured to deliver ablation electric field energy and sensing electrodes, or mapping electrodes, for mapping purposes. In some configurations, the mapping electrodes are configured to be used to collect electrical signals to be used to generate via the operably coupled EAM system 70, and display via the operably coupled display 92, detailed three-dimensional geometric anatomical maps or representations of the cardiac chambers as well as electro-anatomical maps in which cardiac electrical activity of interest is superimposed on the geometric anatomical maps. In some examples, an electrode can operate as an ablation electrode in an ablation mode of the electrophysiology system 50 and as a mapping electrode in a mapping mode of the system 50. Mapping electrodes on the electroporation catheter 105 can measure electrical signals and generate output signals that can be processed by the mapping and navigation controller 90 to generate an electro-anatomical map. In some instances, electro-anatomical maps are generated before ablation for determining the electrical activity of the cardiac tissue within a chamber of interest. In some instances, electro-anatomical maps are generated after ablation in verifying the desired change in electrical activity of the ablated tissue and the chamber. The mapping electrodes may also be used to determine the position of the catheter 105 in three-dimensional space within the body. For example, when the operator moves the distal end of the catheter 105 within a cardiac chamber of interest, the boundaries of catheter movement can be used by the mapping and navigation controller 90 to form the anatomical map of the chamber. The chamber anatomical map may be used to facilitate navigation of the catheter 105 without the use of ionizing radiation such as with fluoroscopy, and for tagging locations of ablations as they are completed in order to guide spacing of ablations and aid the clinician in ablating the anatomy of interest. In some examples, an electrode in the electrode assembly can be configured to only perform an ablation or the electrode in the electrode assembly can be configured to only perform mapping. In some embodiments, however, the sensing electrodes can be configured to deliver ablation energy in addition to the ablation electrodes.

The EAM system 70 is configured to generate the electro-anatomical map for display on the display 92. The EAM system 70 is operable to track the location of the various components of the electroporation catheter system 60, and to generate high-fidelity three-dimensional anatomical and electro-anatomical maps of the heart, including portions of the heart such as cardiac chambers of interest or other structures of interest such as the sinoatrial node or atrioventricular node. In one illustrative example, the EAM system 70 can include the RHYTHMIA™ HDx mapping system marketed by Boston Scientific Corporation. Also, the mapping and navigation controller 90 of the EAM system 70 includes one or more controllers, such as microprocessors or computers, that execute code out of memory to control or perform functional aspects of the EAM system 70, in which the memory, can be part of the one or more controllers, microprocessors, computers, or part of a memory device accessible through a computer network.

The EAM system 70 generates a localization field, via the field generator 80, to define a localization volume about the heart 30, and a location sensor or sensing element on a tracked device, such as sensors on the electroporation catheter 105, generate an output that can be processed by the mapping and navigation controller 90 to track the location of the sensor, and consequently, the corresponding device, within the localization volume. In the illustrated example, the device tracking is accomplished using magnetic tracking techniques, in which the field generator 80 is a magnetic field generator that generates a magnetic field defining the localization volume, and location sensors on the tracked devices are magnetic field sensors.

In other examples, impedance tracking methodologies may be employed to track the locations of the various devices. In such examples, the localization field is an electric field generated, for example, by an external field generator arrangement, such as surface electrodes, by intra-body or intra-cardiac devices, such as an intracardiac catheter, or both. In these examples, the location sensing elements can constitute electrodes on the tracked devices that generate outputs received and processed by the mapping and navigation controller 90 to track the location of the various location sensing electrodes within the localization volume.

The EAM system 70 can be equipped for both magnetic and impedance tracking capabilities. In such examples, impedance tracking accuracy can, in some instances be enhanced by first creating a map of the electric field induced by the electric field generator within the cardiac chamber of interest using a probe equipped with a magnetic location sensor, as is possible using the RHYTHMIA HDx™ mapping system. One exemplary probe is the INTELLAMAP ORION™ mapping catheter marketed by Boston Scientific Corporation.

Regardless of the tracking methodology employed, the EAM system 70 utilizes the location information for the various tracked devices, along with cardiac electrical activity acquired by, for example, the electroporation catheter 105 or another catheter or probe equipped with sensing electrodes, to generate, and display via the display 92, detailed three-dimensional geometric anatomical maps or representations of the heart tissue and voids such as cardiac chambers as well as electro-anatomical maps in which cardiac electrical activity of interest is superimposed on the geometric anatomical maps. Furthermore, the EAM system 70 can generate a graphical representation of the various tracked devices within the geometric anatomical map or the electro-anatomical map.

The electroporation catheter system 60 can be combined or integrated with the EAM system 70 to allow graphical representations of the electric fields that can be produced by the electroporation catheter 105 to be visualized on an electro-anatomical map of the patient's heart. The integrated system can include the capability to enhance the efficiency of clinical workflows, including enhancement of providing a visual representation to the clinician of ablation lesions of portions of the patient's heart created through irreversible electroporation. The integrated system can include generating the graphical representations of the electric fields that can be produced by the electroporation catheter 105, generating the anatomical maps including generating the electro-anatomical maps, and displaying information related to the location and electric field strengths of the electric fields that can be produced by the electroporation catheter 105.

The depiction of the electrophysiology system 50 shown in FIG. 1 is intended for illustration or a general overview of the various components of the system 50 and is not intended to imply that the disclosure is limited to any set of components or arrangement of the components. For example, additional hardware components, such as breakout boxes or workstations, can be included in the electrophysiology system 50.

Catheters with wide area focal ablation electrode assemblies, such as expandable electrode assemblies with an operating radial dimension generally larger than a radial dimension than the catheter shaft, are able to generate larger lesions and footprints on target tissue than catheter shaft electrode and provide for more control and flexibility than singe shot electrode assemblies once the choice in pulmonary vein isolation and posterior wall isolation. Physicians appreciate working with wide area focal ablation catheters if the lesion footprint can be predictable. Further, wide area focal ablation electrode assemblies are often operable in both a monopolar and bipolar mode, which modes create lesions of different morphologies. In some examples, however, wide area focal ablation electrode assemblies designed for both monopolar and bipolar operation are optimized for one mode or the other. For example, a electrode assembly may create preferable wide, omnidirectional electric fields in a monopolar mode may risk arcing in the bipolar mode. This disclosure includes embodiments of catheters that address design considerations for both monopolar and bipolar operation.

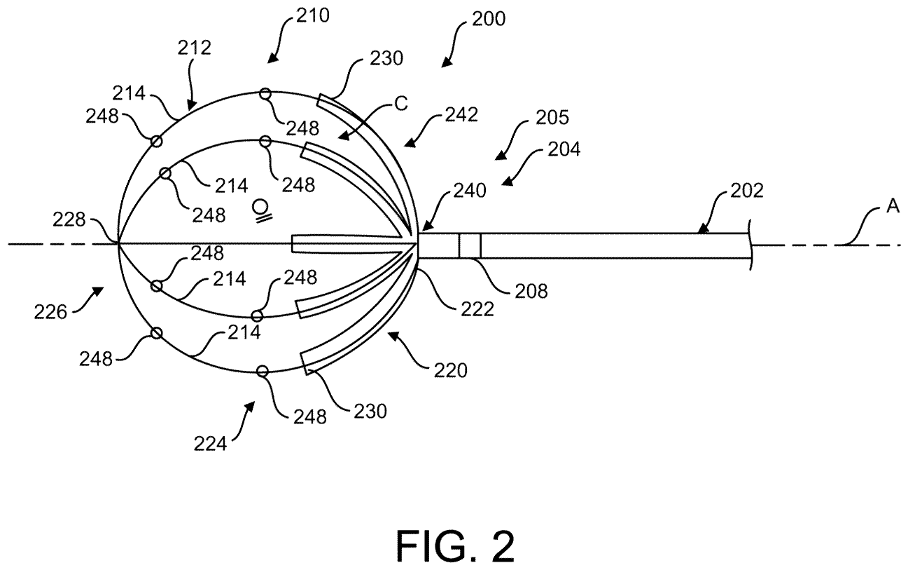

FIG. 2 illustrates features of an electroporation catheter 200, or the distal region 205 of the electroporation catheter 200, for ablation of cardiac tissue, which can be an example of catheter 105 and used with the electrophysiology system 50. The electroporation catheter 200 includes an elongated shaft 202 defining a longitudinal axis A and having distal end 204 opposite a proximal end. The longitudinal axis A is presented as a line passing through a centroid of a cross section of the shaft 202. A distal region 205 of the catheter 200 is configured to be deployed proximate target tissue, such as within a chamber of the patient's heart. An expandable electrode assembly 210 is coupled to the shaft distal end 204 and configured to generate an electric field. In one example, the electric field effects an irreversible electroporation in selected cardiac tissue. The electrode assembly 210 is transitionable between the collapsed state and the expanded state. In the expanded state, the electrode assembly 210 in the expanded state includes a maximum radial dimension greater than a maximum radial dimension of the distal end 204 of the elongated shaft 202. The electrode assembly 210 includes a conductive hull 212 comprising a plurality of conductive struts 214. Embodiments of the conductive hull 212 and struts 214 can be formed in one of a number of spherical configurations. Spherical, as used in this disclosure to define the shape of the electrode assembly in the expanded state, includes spheres, spheroids (as an approximate spherical body), ellipsoids, and other three-dimensional analogues of curvilinear shapes with or without circular symmetry. Among these configurations include a mesh spherical hull formed of a lattice of struts. Another of these configurations include a basket hull formed of struts configured as longitudinally extending splines. Other configurations are contemplated. The hull 212 includes a proximal portion 220 including a proximal end 222 coupled to the elongated shaft 202. The hull 212 extends distally along the axis A to a central portion 224, which includes the maximum radial dimension, and a distal portion 226 converging at a distal tip 228. An insulator 230 is disposed on at least the proximal portion 220 of the hull 212 and surrounds the conductive struts 214. The insulator 230 continuously extends from the proximal end 222 to the medial portion 224.

In embodiments, the elongate shaft 202 is formed of a biocompatible material that provide sufficient sturdiness and flexibility to allow the shaft 202 to be navigated through the vasculature of a patient and reach the treatment site, such as a chamber of the heart. In some embodiments, the shaft 202 is formed of multiple different materials to provide the electroporation catheter 200 with more flexibility at the distal end 204 than a proximal end. Further, the shaft 202 can included a tubular woven member to provide torsional stiffness and bending flexibility. The shaft 202 can include various markers for use with a visualization system, such as radiopaque or echogenic markers, or EAM electrodes to facilitate visualization. The catheter shaft 202 can also accommodate pull wires to deflect the electrode assembly 210 to the treatment site. The distal end 204 can include sensors such as tracking sensors and force sensors and additional elements such as an irrigation element. In some embodiments, a stem (not show) is included extending from the elongate shaft into hull 212 of the ablation electrode, 210, and the stem is configured to include irrigation elements and sensors and other components. In some embodiments, the distal end 204 of the shaft 202 includes an exposed shaft electrode 208, such as plurality of exposed shaft electrodes, proximate the electrode assembly 210. The exposed shaft electrode 208 in embodiments can be used with the electrode assembly 210 and configured as a return electrode in a bipolar mode or configured as an additional active electrode with the electrode assembly 210 to affect in the electric field as described in PFA vectors in a monopolar mode.

The electrode assembly 210 in embodiments includes a conductive coupling portion 240 and a conductive deformable portion 242. The coupling portion 240 is secured to the distal end 204 of the shaft 202, and the deformable portion 242 extends distally from the coupling portion 240. The deformable portion 242 can be collapsed for delivery, such as through an introducer sheath, and expanded for treatment at the treatment site. The coupling portion 240 is mechanically coupled, either directly or indirectly, to the catheter shaft 202. The coupling portion 240 can include components to directly coupled to the shaft 202 or coupled to a transitional part coupled to the shaft 202. The coupling portion 240 is electrically coupled to the conductive paths, such as wires, extending along the shaft 202 to the shaft proximal end. In use with electrophysiology system 50, the electroporation console 130 is electrically coupled via the conductive paths in the shaft 202 to the coupling portion 240. The deformable portion 242 includes the proximal portion 220 mechanically and electrically coupled to the coupling portion 240. In embodiments, the deformable portion 242 defines open area through which blood or other fluids can flow. For example, the hull 212 forms a concave shape around an internal cavity C, and the configuration of the struts 214 provide for openings O in the hull 212. The hull provides a conductive structure that can be configured with system 50 as a single ablation electrode. The hull 212 receives electrical energy and is configured to generate the electric field to effect an irreversible electroporation in selected cardiac tissue. In embodiments, the struts 214 are axial elements that include thin conductors to form the structure of the hull 212 and define the open area. Examples of struts include flexible round wires, pins, straps of metal, strips, flat wires, and planks. In some embodiments, the hull 212 can be formed from a conductive shape memory material such as a nickel titanium alloy that will expand once a constrictor device applying an axial force is removed or from another conductive material that is expanded and contracted via a controllable mechanism. The deformable portion 242 includes a cross-sectional dimension, such as a dimension taken generally perpendicular to the axis A, that is larger than a cross section dimension of the shaft 202. The electrode assembly 210 can provide for wider lesions via electroporation to the tissue in a shorter period of time to create a pattern of overlapping lesions on the tissue reducing the likelihood of arrhythmogenic gaps, or the tip can facilitate the delivery of more power to provide for deeper lesions than catheter shaft electrodes.

The insulator 230 is an electrical insulator that resists delivery of electrical energy on the hull 212 through the insulator 230. The insulator 230 is disposed on at least the proximal portion 220 of the hull 212 and surrounds the conductive struts 214. For example, the insulator 230 extends completely around the strut 214 axial element in the embodiment and does not provide radial gaps of insulation to expose the conductor of the strut 214. The insulator 230 continuously extends from the proximal end 222 to the central portion 224. For example, the insulator 230 does not include longitudinal gaps to expose the conductor of the strut 214. In some embodiments, the insulator 230 is constructed from a polymer applied to the hull 212 such as via a dip coating or spray coating the hull 212. In some embodiments, the insulator 230 is tubular polymer such as a shrink fit polymer disposed about the struts 212 and attached in place. In some embodiments, the insulator 230 is a metal oxide formed by a manufacturing process-such as physical vapor deposition or physical vapor transport (or type of vacuum deposition), sputtering, or an electrochemical process—to deposit a thin film or coating on the strut 212. In addition to the proximal portion 220, the insulator can be extended distally to the central portion 224, such as distal to the maximum radial dimension of the hull 212.

In some embodiments, the electrode assembly 210 includes one or more additional electrodes, such as sensing or mapping electrodes 248 in addition to the conductive hull 212. For example, a plurality of sensing electrodes 248 can be attached to the hull 212 at selected locations, such as the distal tip 228 and electrically isolated from the struts 214. In an example of a basket hull formed of a plurality of splines, the sensing electrodes 248 can be spaced apart from each other each spine. An electrical insulator can be disposed between the sensing electrodes 248 and the associated strut 214. The sensing electrodes 248 are configured for, among other things, sensing cardiac electrical signals, localization of the electrode assembly 210 within the patient anatomy such as via the EAM system 70, and to determine proximity to target tissue within the anatomy. The sensing electrodes 248 in the electrode assembly 210 can be electrically coupled to a one or more lead conductors that extends the length of the shaft 202 that are configured to carry an electrical signal received at the mapping electrode 248. In some embodiments, however, the one or more of the sensing electrodes 248 can be configured with the system 50 to deliver ablation energy in addition to the hull 212.

In embodiments, the catheter 200 is configured to be operated in a bipolar mode to generate an electric field for electroporation. During bipolar operation of the catheter 200, the hull 212 is configured as the anode (or cathode) and a second set of one or more electrodes at the distal portion 205 of the catheter 200, such as exposed shaft electrode 208, is configured as the cathode (or anode), to generate an electrical field. In this example, a pad dispersive electrode described above is not used, and the electrical field is not typically extended through the patient's body, but rather through a localized portion of tissue proximate the electrode assembly 210.

In cases in which the entire hull 212 was exposed and used to create a wide lesion in the target tissue, the bipolar field vector is close to the return electrodes of shaft electrode 208, and the catheter 200 risks arcing and other performance degradations. Additionally, the depth of the lesion in the target tissue is based on whether the exposed surface areas of the active electrode and the return electrode are similar, or, whether the surface area of the tissue contacting active electrode is smaller than the return electrode. Having a fulling exposed hull 212 does not facilitate an optimized bipolar operation. The insulator 230 disposed on the hull focuses electroporation energy rather than projects it symmetrically in all directions radially from spherical hull 212. Accordingly, the insulator 230 is disposed on the hull, particularly at the proximal portion 220 and further distally, to reduce the likelihood of arcing, as well as additional coverage as explained further.

In contrast with bipolar operation, a fully exposed hull 210 is acceptable for operation in monopolar mode. In embodiments, the catheter 200 is also configured to be operated in a monopolar mode to generate an electric field for electroporation. For example, the hull 212 can be electrically coupled to a single lead conductor that extends the length of the shaft 202, or to a set of lead conductors in the shaft 202 that are configured to carry the same electrical signal to generate the electric field. The hull 212 is configured as one of the anode or the cathode. None of the electrodes in the electrode assembly 210 or the distal end 204 of the catheter shaft 202 are configured as the other of the cathode or the anode. Instead, the other of the cathode or the anode is provided in the form of a pad dispersive electrode located on the patient during electroporation. The electrical field is formed between the hull and any other activated electrodes of the electrode assembly 210 or distal end 204 and the pad dispersive electrode.

Via the application of the insulator 230 on the hull 212 to enhance performance in of the catheter 200 in bipolar mode, the electrode assembly 210 projects a suboptimal asymmetrical electric field in the monopolar mode that can vary based on the orientation of the hull 212 with target tissue. In embodiments, the exposed shaft electrode 208 is also configured to carry the same electrical signal to generate the electric field when the catheter is configured to be operated in the monopolar mode. For example, the lead conductors in the shaft 202 electrically coupled to the exposed shaft electrode 208 are configured to carry the same electrical signal as provided to the hull 212 to generate the electric field. The result is a more omnidirectional electric field in the monopolar mode, particularly relative to the distal tip 228.

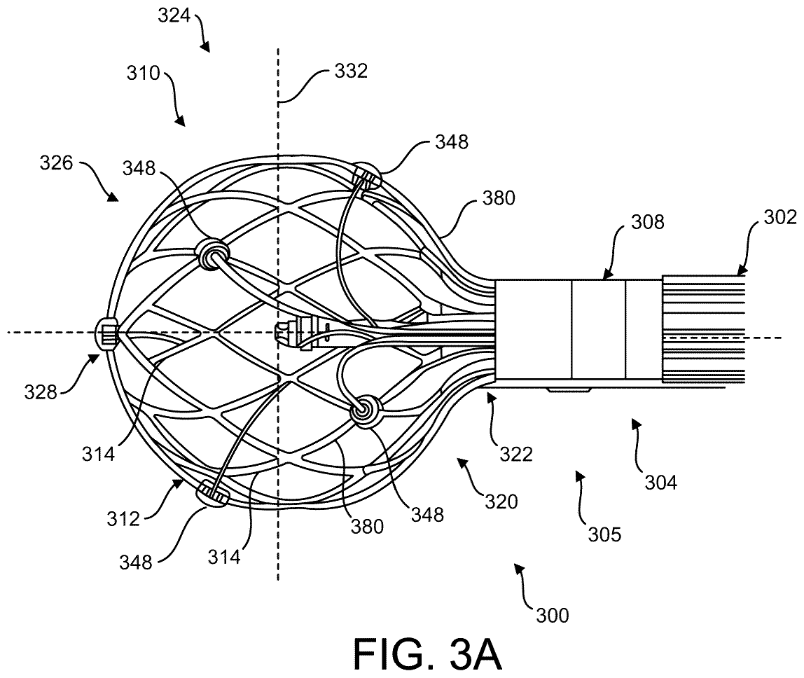

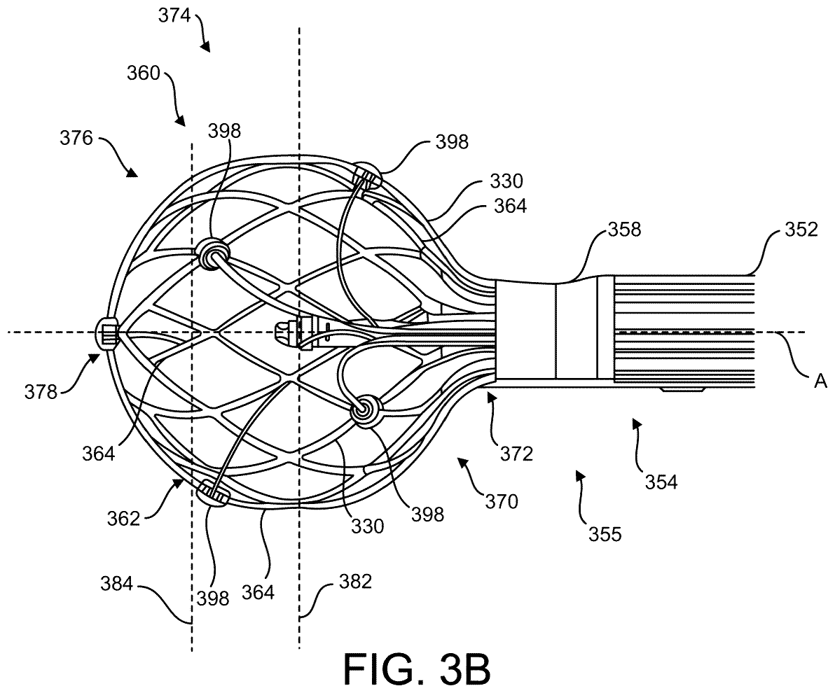

FIGS. 3A and 3B illustrate electroporation catheters 300, 350, or distal portions 305, 355 of electroporation catheters, two embodiments which are constructed in accordance with catheter 200. Catheters 300, 350 each include an elongated shaft 302, 352, respectively, having a proximal end and an opposite distal end 304, 354 defining an axis A. In the embodiments, shafts 302, 352 each include an exposed electrode 308, 358, respectively, such as a ring electrode. The distal regions 305, 355, are configured to be deployed proximate target tissue. Each catheter 300, 350 includes an electrode assembly 310, 360, respectively, coupled to and extending distally from the distal end 304, 354 of the respective elongated shaft 302, 352. The electrode assemblies 310, 360 are each transitionable between a collapsed state and an expanded state. The electrode assemblies 310 and 360 are in the expanded state in the illustrated embodiments. The electrode assemblies 310, 360 include a conductive hull 310, 360 comprising a plurality of conductive struts 312, 362, respectively, defining a spherical shape in the expanded state. The hulls 312, 362 include a proximal portion 320, 370, respectively, including a proximal end 322, 372 coupled to the elongated shaft 302, 352, respectively. The hulls 312, 362, respectively extend distally along the axis A to a respective central portion 324, 374, which include the maximum radial dimension, and a distal portion 326, 376 converging at a distal tip 328, 378, respectively. An insulator 330, 380 is disposed on at least the proximal portions 320, 370 of the hulls 312, 362, respectively, and surrounds the conductive struts 314, 364, respectively. The insulator 330, 380 continuously extend from the proximal end 322, 372 to the medial portions 324, 374, respectively.

Electroporation catheters 300, 350 illustrate generally spherical hulls 312, 362, respectively, formed from a plurality of interconnected struts 314, 364, respectively. The struts 314, 364, are arranged in plurality of cells coupled together at joints on the hulls, 312, 362, respectively that form a lattice. The lattice is expandable and collapsible and expands into the operable, spherical form in the expanded configuration. In embodiments, the lattice struts 314, 364 are constructed from a conductive, shape-memory material, and the lattice operates as a single ablation electrode of the electrode assembly 310, 360. In some embodiments, the lattice struts and cells are arranged symmetrically around the hull. In other embodiments, the lattice struts are unequally distributed around the hull to provide for more conductive surface nearer the distal portion 326, 376, respectively. In the illustrated embodiments, the electrode assemblies 310, 360 include sensing electrodes 348, 398 attached to the lattice hulls 312, 362, respectively, and a distal tip electrode 349, 399, respectively, that can be configured as a sensing electrode in some embodiments.

The insulator 330, 380 is disposed on at least the proximal portions 320, 370 of the lattice hulls 312, 362, respectively, and surrounds the conductive struts 314, 364, respectively. The insulator 330, 380 continuously extend from the proximal end 322, 372 to the medial portions 324, 374, respectively. In one embodiment, FIG. 3A illustrates the insulator 330 is disposed on the conductive struts 314 of the electrode assembly 310 from the shaft 304 to a hull equator 332, which is congruent with the maximum dimension (diameter) of the electrode assembly 310 as measured perpendicularly from the axis A. The conductive surfaces of the struts 314 proximal to the equator 332 are completely surrounded by the insulator 330. The conductive struts 314 distal to the equator 332 are exposed, and the conductive struts proximal to the equator 332 are surrounded by insulator 330 in the illustrated example. In another embodiment, FIG. 3B illustrates the insulator 380 is disposed on the conductive struts 364 of the electrode assembly 360 from the shaft 354 to distal the hull equator 382 to a chord 384 in a plane perpendicular to the axis A. The conductive struts 364 distal to the chord 384 are exposed, and the conductive struts proximal to the chord 384 are surrounded by insulator 330 in the illustrated example.

In a set of examples, the electroporation catheters 300, 350 were configured for bipolar operation and compared to a catheter with no insulation on the conductive struts on the hull. During bipolar operation of the catheters, the hulls were configured as either the anode or the cathode and a second set of one or more electrodes at the distal portion of the catheter, such as exposed shaft electrode, were configured as the other of the cathode and the anode, to generate an electrical field via an electric pulse.

In a first set of examples, the expanded hulls of the three catheters each had an 8-millimeter diameter and applied a 2-kilovolt pulse. In the example of the catheter having an 8-mm diameter hull with no insulation, the predicted lesion width was 17-mm and the predicted lesion depth was 5.0-mm. In the example of the catheter 300 having an 8-mm diameter hull 312 proximal to the equator 332 covered in insulation 330 and the conductive struts 314 distal to the equator 332 exposed, the predicted lesion width was 18-mm and the predicted lesion depth was 6.0-mm. In the example of the catheter 350 having an 8-mm diameter hull 362 proximal to the chord 382 covered in insulation 380 and the conductive struts 364 distal to the chord exposed, with approximately two-thirds of the hull 362 covered in insulation 380, the predicted lesion width was 19-mm and the predicted lesion depth was 6.6-mm.

In a second set of examples, the expanded hulls of the three catheters each had a 7-mm diameter and applied a 2-kilovolt pulse. In the example of the catheter having a 7-mm diameter hull with no insulation, the predicted lesion width was 17-mm and the predicted lesion depth was 5.5-mm. In the example of the catheter 300 having a 7-mm diameter hull 312 proximal to the equator 332 covered in insulation 330 and the conductive struts 314 distal to the equator 332 exposed, the predicted lesion width was 18-mm and the predicted lesion depth was 6.1-mm. In the example of the catheter 350 having a 7-mm hull 362 proximal to the chord 382 covered in insulation 380 and the conductive struts 364 distal to the chord exposed, with approximately two-thirds of the hull 362 covered in insulation 380, the predicted lesion width was 18-mm and the predicted lesion depth was 6.8-mm.

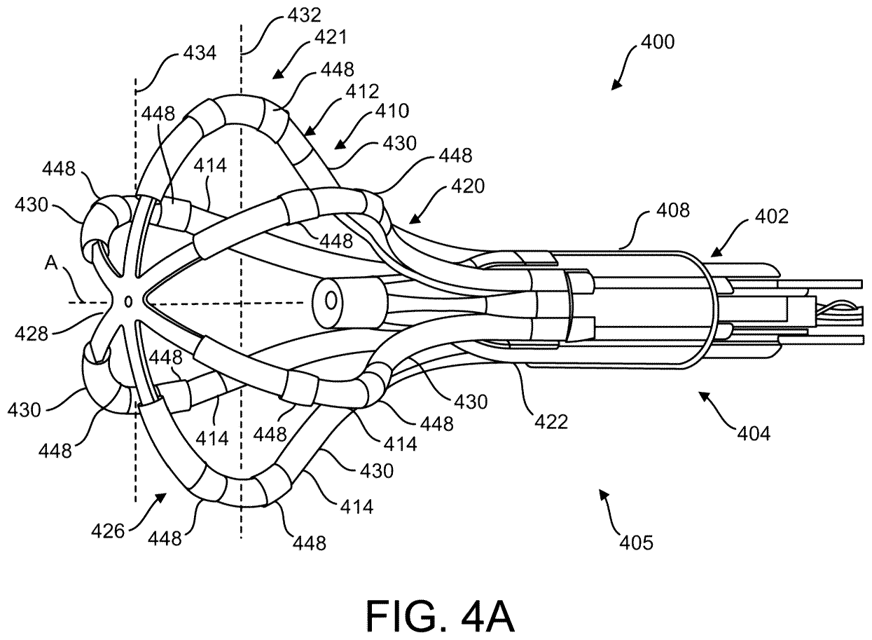

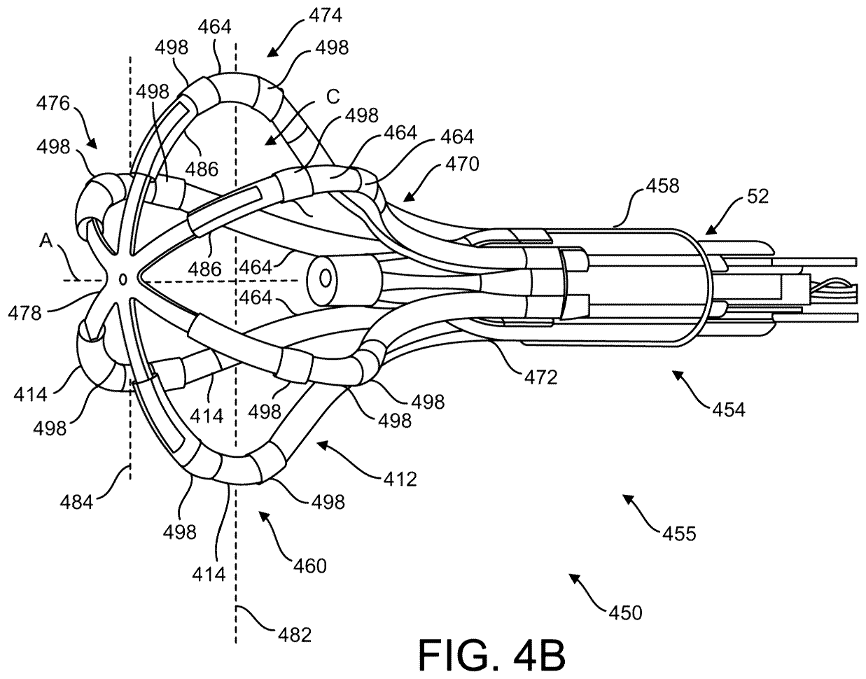

FIGS. 4A and 4B illustrate electroporation catheters 400, 450, or distal portions 405, 455 of electroporation catheters, two embodiments which are constructed in accordance with catheter 200. Catheters 400, 450 each include an elongated shaft 402, 452, respectively, having a proximal end and an opposite distal end 404, 454 defining an axis A. In the embodiments, shafts 402, 452 each include an exposed electrode 408, 458, respectively, such as a ring electrode. The distal regions 405, 455, are configured to be deployed proximate target tissue. Each catheter 400, 450 includes an electrode assembly 410, 460, respectively, coupled to and extending distally from the distal end 404, 454 of the respective elongated shaft 402, 452. The electrode assemblies 410, 460 are each transitionable between a collapsed state and an expanded state. The electrode assemblies 410 and 460 are in the expanded state in the illustrated embodiments. The electrode assemblies 410, 460 include a conductive basket 410, 460 comprising a plurality of conductive struts configured as splines 412, 462, respectively, defining a spherical shape in the expanded state. The baskets 412, 462 include a proximal portion 420, 470, respectively, including a proximal end 422, 472 coupled to the elongated shaft 402, 452, respectively. The baskets 412, 462, respectively extend distally along the axis A to a respective central portion 424, 474, which include the maximum radial dimension, and a distal portion 426, 476 converging at a distal tip 428, respectively. An insulator 430, 480 is disposed on at least the proximal portions 420, 470 of the baskets 412, 462, respectively, and surrounds the conductive struts 414, 464, respectively. The insulator 430, 480 continuously extend from the proximal end 422, 472 to the medial portions 424, 474, respectively.

Electroporation catheters 400, 450 illustrate generally spherical basket hulls 412, 462, respectively, formed from a plurality of struts configured as longitudinally extending splines 414, 464, respectively. The basket 412, 462 is expandable and collapsible and expands into the operable, spherical form in the expanded configuration. In embodiments, the splines 414, 464 are constructed from a conductive, shape-memory material, and the struts of the basket operate as a single ablation electrode of the electrode assembly 410, 460. In some embodiments, the splines are arranged symmetrically around the basket. In other embodiments, the position of the splines are unequally distributed around the basket to provide for more conductive surface nearer one side of the basket. In the illustrated embodiments, the electrode assemblies 410, 460 include sensing electrodes 448, 498 attached to the splines 414, 464, respectively, and a distal tip electrode 449, 499, respectively, that can be configured as a sensing electrode in some embodiments.

The insulator 430, 480 is disposed on at least the proximal portions 420, 470 of the spines 414, 464 of the baskets 412, 462, respectively, and surrounds the conductive splines 414, 464, respectively. The insulator 430, 480 continuously extend from the proximal end 422, 472 to the medial portions 424, 474, respectively. In one embodiment, FIG. 4A illustrates the insulator 430 is disposed on the conductive splines 414 of the electrode assembly 410 from the shaft 404 and distal to a basket equator 432, which is congruent with the maximum dimension (diameter) of the electrode assembly 410 as measured perpendicularly from the axis A to a chord 434 in a plane perpendicular to the axis A. The conductive surfaces of the splines 414 proximal to the chord 434 are completely surrounded by the insulator 430. The conductive splines 414 distal to the chord 434 are exposed, and the conductive splines proximal to the chord 434 are surrounded by insulator 430 in the illustrated example.

In another embodiment, FIG. 4B illustrates the insulator 480 is disposed on the conductive splines 464 of the electrode assembly 460 from the shaft 454 to the basket equator 482 in a plane perpendicular to the axis A. The conductive struts proximal to the equator 482 are surrounded by insulator 430 in the illustrated example. Another insulator, or second insulator 486 is selectively disposed on the splines 464 distal to the equator 484 to selectively shape the electric field. For example, the second insulator 486 can be applied to portions of the splines 464 facing an inner cavity C, such as distal from the equator 482 to a chord 484 in a plane perpendicular to the axis A and distal from the equator 482. In embodiments, the second insulator 486 can be applied to some or all of the inner surfaces of the splines 464, i.e., the surfaces facing the inner cavity C. In embodiments, the conductive surfaces of the splines 464 distal to the chord 484 are complexly exposed. Various configurations of the second insulator 486 in combination with the insulator 480 and the exposed portions of the splines 464 distal to the equator 482 are contemplated.

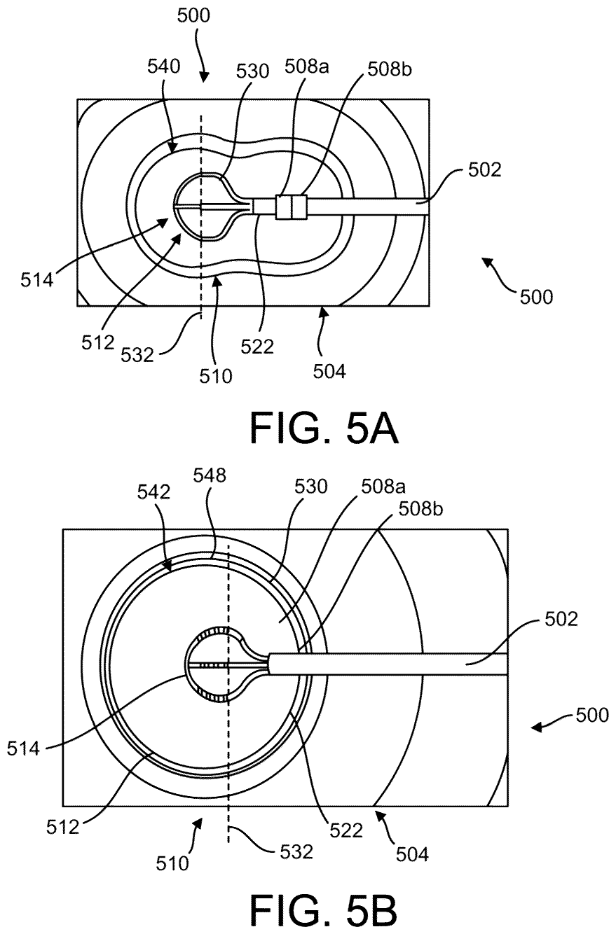

FIGS. 5A and 5B illustrate two examples of the electric field vectors produced by a basket catheter 500. The basket catheter 500 includes include an elongated shaft 502 having a proximal end and an opposite distal end 504 defining an axis A. In the embodiment, shaft 502 include an exposed electrode 508, such as electrode 508a, 508b. The catheter 500 includes an electrode assembly 510 coupled to and extending distally from the distal end 504 of the elongated shaft 502. The electrode assembly 510 is in the expanded state in the illustrated embodiments. The electrode assemblies 510 includes a conductive basket 512 comprising a plurality of conductive struts configured as splines 514 defining a spherical shape in the expanded state. An insulator 530 is disposed on the basket 512 and surrounds the conductive splines 514 from a proximal end 522 to a basket equator 532. The insulator 530 continuously extends from the proximal end 522 to the equator 532.

FIG. 5A illustrates the electric field vectors 540 produced via the catheter 500 operated in a bipolar mode. During bipolar operation of the catheter 500, the basket 512 is configured as the anode (or cathode) and the exposed shaft electrodes 508a and 508b are configured as the cathode (or anode), to generate an electrical field. The insulator 530 disposed on the basket 512 focuses electroporation energy rather than projects it symmetrically in all directions radially from spherical basket 512. Accordingly, the insulator 530 is disposed on the splines, particularly at the proximal portion 220 and further distally to the equator 532, to reduce the likelihood of arcing.

FIG. 5B illustrates the electric field vectors 542 produced via the catheter 500 operated in a monopolar mode. The basket 512 is configured as one of the anode or the cathode. None of the electrodes in the electrode assembly 510 or the distal end 504 of the catheter shaft 502 are configured as the other of the cathode or the anode. Instead, the other of the cathode or the anode is provided in the form of a pad dispersive electrode. The electrical field is formed between the hull and any other activated electrodes of the electrode assembly 510 or distal end 504 and the pad dispersive electrode. Via the application of the insulator 530 on the hull 212 to enhance performance in of the catheter 500 in bipolar mode, the electrode assembly 510 projects a suboptimal asymmetrical electric field in the monopolar mode that can vary based on the orientation of the hull 512 with target tissue. In embodiments, the exposed shaft electrodes 508a, 508b are also configured to carry the same electrical signal to generate the electric field when the catheter 500 is configured to be operated in the monopolar mode. If the electrode assembly includes sensing electrodes 548 coupled to the basket 512, the sensing electrodes 548 can be activated with the same electrical signal to generate the electric field in the splines 514 to selectively shape the electric field vectors 542. The result is a more omnidirectional electric field in the monopolar mode.

Various modifications and additions can be made to the exemplary embodiments discussed without departing from the scope of the present disclosure. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. An electroporation catheter for ablation of cardiac tissue, the electroporation catheter comprising:
an elongated shaft having a proximal end and an opposite distal end, the elongated shaft defining an axis;
an electrode assembly coupled to and extending distally from the distal end of the elongated shaft, the electrode assembly transitionable between a collapsed state and an expanded state wherein the electrode assembly includes a plurality of conductive struts defining a spherical shape in the expanded state having a proximal portion coupled to the elongated shaft, a central portion having a maximum radial dimension and a distal portion opposite the elongated shaft; and
a proximal insulation portion disposed on the proximal portion of the electrode assembly surrounding each of the plurality of conductive struts, the proximal insulator portion extending from the proximal end to at least the central portion.

2. The electroporation catheter of claim 1, wherein the ablation electrode in the expanded state includes maximum radial dimension greater than a maximum radial dimension of the distal region of the elongated shaft.

3. The electroporation catheter of claim 2, wherein the maximum radial dimension of the ablation electrode defines an equator on the electrode assembly.

4. The electroporation catheter of claim 3, wherein the proximal insulation is disposed on the conductive struts from the proximal end to the equator.

5. The electroporation catheter of claim 3, wherein the proximal insulator is disposed on the conductive struts from the proximal end to a chord on the electrode assembly distal to the equator.

6. The electroporation catheter of claim 1, wherein the conductive struts form a lattice hull.

7. The electroporation catheter of claim 6, wherein the conductive struts are arranged in a plurality of cells on the lattice hull.

8. The electroporation catheter of claim 1, wherein the conductive struts include a plurality of longitudinally extending splines to form a basket hull.

9. The electroporation catheter of claim 1, further comprising a shaft electrode disposed on the distal region of the shaft.

10. The electroporation catheter of claim 9, wherein the electrode assembly and shaft electrode are configured as one of a cathode and an anode to generate an electric field in a monopolar mode.

11. The electroporation catheter of claim 9, wherein the electrode assembly is configured as one of a cathode and an anode and the shaft electrode is configured as the other of the anode and the cathode to generate an electric field in a bipolar mode.

12. The electroporation catheter of claim 1, further comprising a plurality of sense electrodes disposed on the electrode assembly.

13. The electroporation catheter of claim 12, wherein the electrode assembly includes a plurality of electrodes configured as one of a cathode and an anode to generate an electric field in a monopolar mode.

14. The electroporation catheter of claim 1, further comprising a second insulator selectively disposed on the conductive struts distal to the proximal insulation portion.

15. The electroporation catheter of claim 14, wherein the second insulator is disposed on an inner surface of a strut of the plurality of conductive struts facing an internal cavity of the electrode assembly, and an outer surface of the strut of the plurality of conductive struts is exposed.

16. An electrophysiology system, comprising:
an electroporation console configured to generate pulsed electrical signals for electroporation ablation; and
an electroporation catheter coupled to the electroporation console, the electroporation catheter comprising:
an elongated shaft having a proximal end and an opposite distal end, the elongated shaft defining an axis:
an electrode assembly coupled to and extending distally from the distal end of the elongated shaft, the electrode assembly transitionable between a collapsed state and an expanded state wherein the electrode assembly includes a plurality of conductive struts defining a spherical shape in the expanded state having a proximal portion coupled to the elongated shaft, a central portion having a maximum radial dimension and a distal portion opposite the elongated shaft; and
a proximal insulation portion disposed on the proximal portion of the electrode assembly surrounding each of the plurality of conductive struts, the proximal insulator portion extending from the proximal end to at least the central portion.

17. The electrophysiology system of claim 16, wherein the electroporation catheter is configured to be operated in a bipolar mode and a monopolar mode.

18. An electroporation catheter for ablation of cardiac tissue, the electroporation catheter comprising:
an elongated shaft having a proximal end and an opposite distal end, the elongated shaft defining an axis and including a shaft electrode disposed on the distal end;
an electrode assembly coupled to and extending distally from the distal end of the elongated shaft, the electrode assembly transitionable between a collapsed state and an expanded state wherein the electrode assembly includes a plurality of conductive struts defining a spherical shape in the expanded state having a proximal portion coupled to the elongated shaft, a central portion having a maximum radial dimension and a distal portion opposite the elongated shaft, the electrode assembly operable in a bipolar mode and a monopolar mode; and
a proximal insulation portion disposed on the proximal portion of the electrode assembly surrounding each of the plurality of conductive struts, the proximal insulator portion extending from the proximal end to at least the central portion;
wherein the electrode assembly and shaft electrode are configured as one of a cathode and an anode to generate an electric field in the monopolar mode, and
wherein the electrode assembly is configured as one of a cathode and an anode and the shaft electrode is configured as the other of the anode and the cathode to generate an electric field in the bipolar mode.

19. The electroporation catheter of claim 18, wherein the ablation electrode in the expanded state includes maximum radial dimension greater than a maximum radial dimension of the distal region of the elongated shaft, and wherein the proximal insulator is disposed on the conductive struts from the proximal end to a chord on the electrode assembly distal to the maximum radial dimension.

20. The electroporation catheter of claim 18, wherein the conductive struts form one of a lattice hull and a basket hull.
